(19) United States
(12) Patent Application Publication
(10) Pub. No.: US 2019/0183428 A1
(43) Pub. Date: Jun. 20, 2019

(54) METHOD AND APPARATUS FOR APPLYING MACHINE LEARNING TO CLASSIFY PATIENT MOVEMENT FROM LOAD SIGNALS

(71) Applicant: Hill-Rom Services, Inc., Batesville, IN (US)

(72) Inventors: Yongji FU, Harrison, OH (US); Chunhui ZHAO, Hangzou (CN); Shengwei LUO, Hangzou (CN)

(21) Appl. No.: 16/224,020

(22) Filed: Dec. 18, 2018

Related U.S. Application Data
(60) Provisional application No. 62/695,392, filed on Jul. 9, 2018.
(60) Provisional application No. 62/607,572, filed on Dec. 19, 2017.

(51) Int. Cl. A61B 5/00; A61B 5/11; A61G 7/05

(52) U.S. Cl. CPC A61B 5/7264; A61B 5/1115; A61B 5/6892; A61G 2203/44; A61G 7/05; A61B 2562/0252; A61B 5/7203

(57) ABSTRACT

Neural network approaches to identify the action of bedridden patients and consider whether they have made a particular movement is disclosed. The inputs to the embodiments of the neural networks are four time series signals acquired from load cells placed in the four corners of the bed. Through the network, the corresponding memberships of pre-defined actions are obtained.

### PRIORITY CLAIM

This application claims priority under 35 U.S.C. § 119(e) to US Provisional Application Nos. 62/695,392, filed Jul. 9, 2018, and 62/607,572, filed Dec. 19, 2017, which are expressly incorporated by reference herein.

### BACKGROUND

The present disclosure relates to a method and apparatus for creating a sensor system that makes a real-time determination of the type of movement a patient is making on a patient support apparatus and responding to that movement to make an automatic intervention.

Known systems employ various sensors to detect the location of a patient on a patient support apparatus and predict patient activities based on real time signals from load sensors of the patient support apparatus. In general, these systems are limited to classifying the in-bed patient activity into two classes: exiting the bed or not. That is to say, other actions like turning over and sitting up in bed are difficult or impossible to be recognized. Thus these undefined actions will quite possibly be misclassified into exiting due to the high sensitivity. False alarms are therefore generated which will not only create unnecessary distractions but also cause alarm fatigue on the part of caregivers so that critical alarms are likely to be missed by the staff.

The main issues in activity recognition are described as below: Machine learning algorithm has been used for a long time, it may not work well sometimes. Machine learning systems often need a feature extraction which may be inaccurate. As for deep learning algorithms, though they work very well, deep learning structures are becoming more and more complex. As the structure becomes more complex, the number of the parameters needed to train or set also gets larger. Thus it consumes time when the model needs to be retrained for both two learning method.

### SUMMARY

The present disclosure includes one or more of the features recited in the appended claims and/or the following features which, alone or in any combination, may comprise patentable subject matter.

A patient support apparatus is configured to operate as a sensing device to characterize patient movement by monitoring sensor signals in real-time data, using a convolution neural network to analyze the date, and applying a probability density function to discriminate the type of movement the patient is making from a predefined set of movements.

According to a first aspect of the present disclosure, a sensing system for detecting, classifying, and responding to a patient action comprises a frame, a plurality of load sensors supported from the frame, a patient supporting platform supported from the plurality of load sensors so that the entire load supported on the patient supporting platform is transferred to the plurality of load sensors, and a controller supported on the frame. The controller is electrically coupled to the load sensors and operable to receive a signal from each of the plurality of load sensors with each load sensor signal representative of a load supported by the respective load sensor. The controller includes a processor and a memory device. The memory device includes a non-transitory portion storing instructions that, when executed by the processor, cause the controller to: capture time sequenced signals from the load cells, input the time sequenced signals to a convolution neural network to establish the membership of an action indicated by the signals, apply a probability density function to the membership determination to establish a confidence interval for the particular membership, and if the confidence is sufficient, provide an indicator identifying the most likely action indicated by the signals.

In some embodiments, the time sequenced signals are filtered using a median filtering applied to predefined groups of time sequenced data points of the signals.

In some embodiments, the filtered data signals are down sampled prior to being input into the convolution neural network.

In some embodiments, the convolution neural network is trained using historical signal data.

In some embodiments, the output of the convolution neural network is limited to either a value of 0 or 1 using the sigmoid function.

In some embodiments, the feature map of a convolution layer output is pooled over a local temporal neighborhood by a sum pooling function.

In some embodiments, a mean square error function is applied as a cost function for the neural network.

In some embodiments, the load signals are normalized based on the patient's weight.

According to a second aspect of the present disclosure, a method of operating a sensing system for detecting, classifying, and responding to a patient action on a patient support apparatus comprises capturing time sequenced signals from load cells supporting a patient, inputting the time sequenced signals to a convolution neural network to establish the membership of an action indicated by the signals, applying a probability density function to the membership determination to establish a confidence interval for the particular membership, and if the confidence is sufficient, providing an indicator identifying the most likely action indicated by the signals.

In some embodiments, the time sequenced signals are filtered using a median filtering applied to predefined groups of time sequenced data points of the signals.

In some embodiments, the filtered data signals are down sampled prior to being input into the convolution neural network.

In some embodiments, the convolution neural network is trained using historical signal data.

In some embodiments, the output of the convolution neural network is limited to either a value of 0 or 1 using the sigmoid function.

In some embodiments, the feature map of a convolution layer output is pooled over a local temporal neighborhood by a sum pooling function.

In some embodiments, a mean square error function is applied as a cost function for the neural network.

In some embodiments, the load signals are normalized based on the patient's weight.

According to another aspect of the present disclosure, a sensing system for detecting, classifying, and responding to a patient action comprises a frame, a plurality of load sensors supported from the frame, a patient supporting platform supported from the plurality of load sensors, and a controller supported on the frame. The controller is electrically coupled to the load sensors and operable to receive a signal from each of the plurality of load sensors with each load sensor signal representative of a load supported by the respective load sensor. The controller also includes a processor and a memory device, the memory device including a non-transitory portion storing instructions. When the instructions are executed by the processor, it causes the controller to capture time sequenced signals from the load cells, input the time sequenced signals to a broad learning network to establish the classification of an action indicated by the signals, and provide an indicator identifying the most likely action indicated by the signals.

In some embodiments, the time sequenced signals are filtered using a median filtering applied to predefined groups of time sequenced data points of the signals.

In some embodiments, the filtered data signals are down sampled prior to being input into the broad learning network.

In some embodiments, the broad learning network includes a sparse auto encoder for feature extraction.

In some embodiments, the broad learning network includes a random vector functional-link neural network for classification of the action.

In some embodiments, the sparse auto-encoder utilizes a sigmoid function to determine the activation of the neurons of the neural network.

In some embodiments, the sparse auto-encoder utilizes a tangent function to determine the activation of the neurons of the neural network.

In some embodiments, the sparse auto-encoder utilizes the Kullback-Leibler divergence method to determine the activation of the neurons of the neural network.

In some embodiments, the random vector is determined by gradient descent.

In some embodiments, enhancement nodes of the neural network are determined using randomly generated weights on the feature map.

In some embodiments, the pseudoinverse of the feature matrix is determined by a convex optimization function.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view from the foot end on the patient's right of a patient support apparatus;

FIG. 2 is a block diagram of a portion of the electrical system of the patient support apparatus of FIG. 1;

FIG. 3 is a diagrammatic representation of the positions of a number of load cells of the patient support apparatus of FIG. 1;

FIG. 4 is a diagrammatic representation of a filtered sample of the signals from each of the load cells with the region encircled indicating the signal during a movement by an occupant of the patient support apparatus;

FIG. 5 is a diagrammatic representation of the signals of FIG. 4 after a down sampling process has been applied to the signals;

FIG. 6 is a diagrammatic representation of the machine learning model applied to the signals from the load cells;

FIG. 7 is a chart showing the error convergence as a function of the number of iterations applied in the learning model of FIG. 6;

FIG. 8 is a comparison of the probability density functions for the predictions of the membership classification of each potential movement from a set of signals;

FIG. 9 is a comparison of the probability density functions for the confidence intervals of each of the probability density functions of FIG. 8;

FIG. 10 is a diagrammatic representation of a model of a functional-link neural network;

FIG. 11 is a graph representing the data signal from the load cells of FIG. 3 during an action being conducted by an occupant of the patient support apparatus of FIG. 2;

FIG. 12 is a diagrammatic representation of a sparse auto encoder of a the present disclosure; and

FIG. 13 is a diagrammatic representation of a broad learning system employing a random variable functional-link neural network according to the present disclosure.

### DETAILED DESCRIPTION

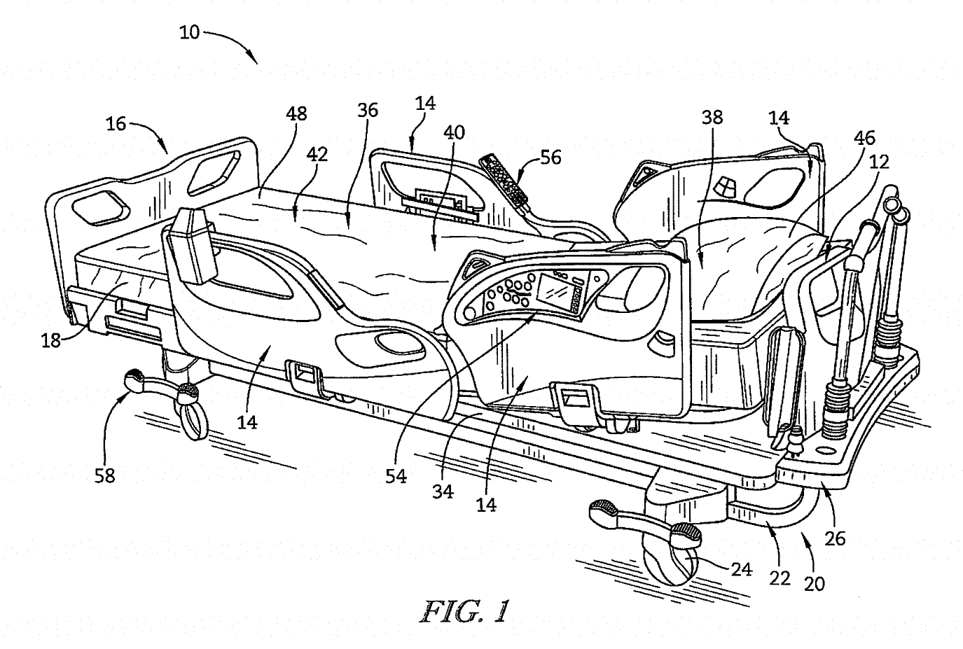

An illustrative patient support apparatus 10 embodied as a hospital bed is shown in FIG. 1. The patient support apparatus 10 of FIG. 1 has a fixed bed frame 20 which includes a stationary base frame 22 with casters 24 and an upper frame 26. The stationary base frame 22 is further coupled to a weigh frame 30 that is mounted via frame member 32a and 32b to an adjustably positionable mattress support frame or deck 34 configured to support a mattress 18. The mattress 18 defines a patient support surface 36 which includes a head section 38, a seat section 40, and a foot section 42. The patient support apparatus 10 further includes a headboard 12 at a head end 46 of the patient support apparatus 10, a footboard 14 at a foot end 48 of the patient support apparatus 10, and a pair of siderails 16 coupled to the upper frame 26 of the patient support apparatus 10. The siderail 16 supports a patient monitoring control panel and/or a mattress position control panel 54. The patient support apparatus 10 is generally configured to adjustably position the mattress support frame 34 relative to the base frame 22.

Conventional structures and devices may be provided to adjustably position the mattress support frame 34, and such conventional structures and devices may include, for example, linkages, drives, and other movement members and devices coupled between base frame 22 and the weigh frame 30, and/or between weigh frame 30 and mattress support frame 34. Control of the position of the mattress support frame 34 and mattress 18 relative to the base frame 22 or weigh frame 30 is provided, for example, by a patient control pendant 56, a mattress position control panel 54, and/or a number of mattress positioning pedals 58. The mattress support frame 34 may, for example, be adjustably positioned in a general incline from the head end 46 to the foot end 48 or vice versa. Additionally, the mattress support frame 34 may be adjustably positioned such that the head section 38 of the patient support surface 36 is positioned between minimum and maximum incline angles, e.g., 0-65 degrees, relative to horizontal or bed flat, and the mattress support frame 34 may also be adjustably positioned such that the seat section 40 of the patient support surface 36 is positioned between minimum and maximum bend angles, e.g., 0-35 degrees, relative to horizontal or bed flat. Those skilled in the art will recognize that the mattress support frame 34 or portions thereof may be adjustably positioned in other orientations, and such other orientations are contemplated by this disclosure.

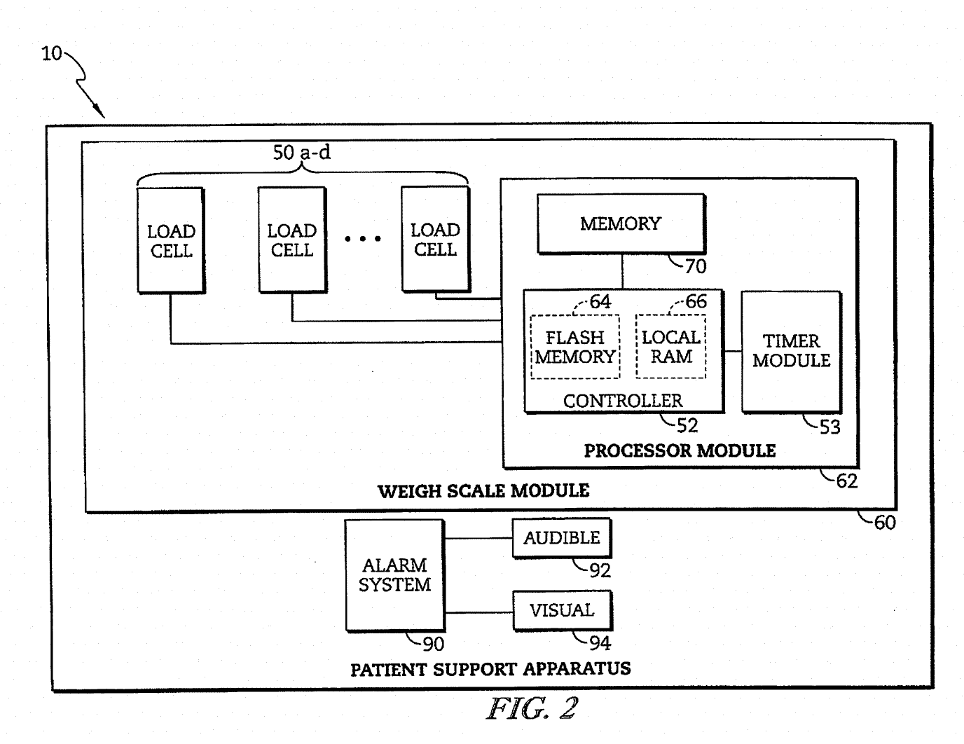

In one illustrative embodiment shown diagrammatically in FIG. 2, the patient support apparatus 10 includes a weigh scale module 60 and an alarm system 90. The weight scale module 60 is configured to determine a plurality set of calibration weights for each of a number of load cells 50 for use in determining a location and an accurate weight of the patient. To determine a weight of a patient supported on the patient support surface 36, the load cells 50 are positioned between the weigh frame 30 and the base frame 22. Each load cell 50 is configured to produce a voltage or current signal indicative of a weight supported by that load cell 50 from the weigh frame 30 relative to the base frame 22. The weigh scale module 60 includes a processor module 62 that is in communication with each of the respective load cells 50. The processor module 62 includes a microprocessor-based controller 52 having a flash memory unit 64 and a local random-access memory (RAM) unit 66. The local RAM unit 66 is utilized by the controller 52 to temporarily store information corresponding to features and functions provided by the patient support apparatus 10. The alarm system 90 is configured to trigger an alarm if the movement of the patient exceeds a predetermined threshold or meets an alarm classification as discussed in further detail below. The alarm may be an audible alarm 92 and/or a visual alarm 94. The visual alarm 94 may be positioned, for example, on the mattress position control panel 54 and/or the patient control pendant 56.

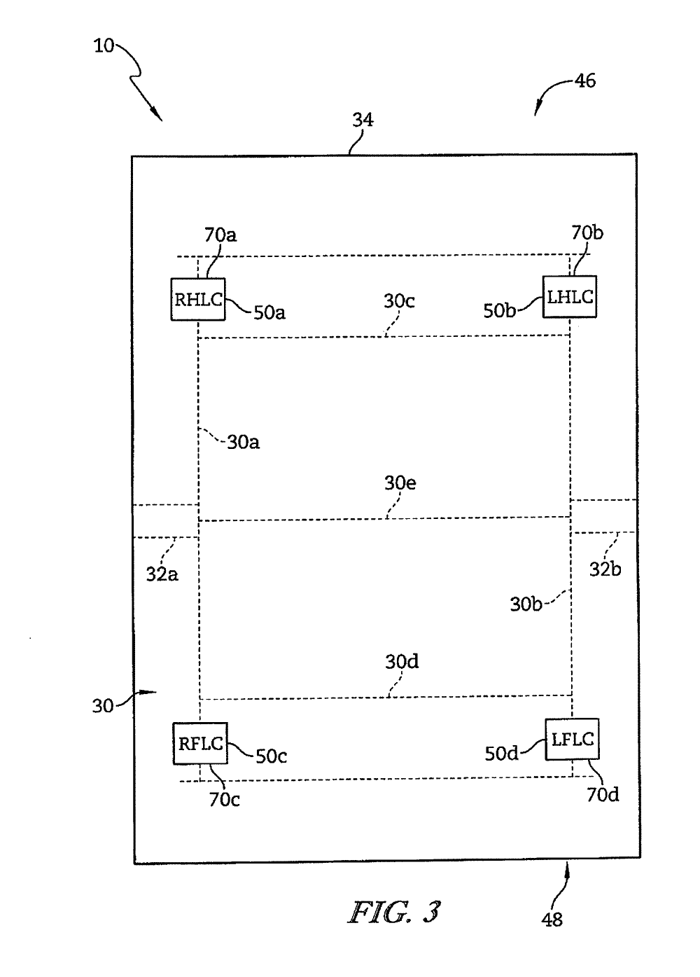

In the illustrated embodiment of FIG. 3, four such load cells 50a-50d are positioned between the weigh frame 30 and the base frame 22; one each near a different corner of the patient support apparatus 10. All four load cells 50a-50d are shown in FIG. 3. Some of the structural components of the patient support apparatus 10 will be designated hereinafter as “right”, “left”, “head” and “foot” from the reference point of an individual lying on the individual's back on the patient support surface 36 with the individual's head oriented toward the head end 46 of the patient support apparatus 10 and the individual's feet oriented toward the foot end 48 of the patient support apparatus 10. For example, the weigh frame 30 illustrated in FIG. 3 includes a head end frame member 30c mounted at one end to one end of a right side weigh frame member 30a and at an opposite end to one end of a left side frame member 30b. Opposite ends of the right side weigh frame member 30a and the left side weigh frame member 30b are mounted to a foot end frame member 30d. A middle weigh frame member 30e is mounted at opposite ends to the right and left side weigh frame members 30a and 30b respectively between the head end and foot end frame members 30c and 30d. The frame member 32a is shown mounted between the right side frame member 30a and the mattress support frame 34, and the frame member 32b is shown mounted between the left side frame member 30b and the mattress support frame 34. It will be understood that other structural support is provided between the weigh frame member 30 and the mattress support frame 34.

A right head load cell (RHLC) 50a is illustratively positioned near the right head end of the patient support apparatus 10 between a base support frame 44a secured to the base 44 near the head end 46 of the patient support apparatus 10 and the junction of the head end frame member 30c and the right side frame member 30a, as shown in the block diagram of FIG. 2. A left head load cell (LHLC) 50b is illustratively positioned near the left head end of the patient support apparatus 10 between the base support frame 44a and the junction of the head end frame member 30c and the left side frame member 30b, as shown in the diagram of FIG. 3. A right foot load cell (RFLC) 50c is illustratively positioned near the right foot end of the patient support apparatus 10 between a base support frame 44b secured to the base 44 near the foot end 48 of the patient support apparatus 10 and the junction of the foot end frame member 30d and the right side frame member 30a, as shown in the diagram of FIG. 3. A left foot load cell (LFLC) 50d is illustratively positioned near the left foot end of the patient support apparatus 10 between the base support frame 44b and the junction of the foot end frame member 30d and the left side frame member 30b. In the exemplary embodiment illustrated in FIG. 3, the four corners of the mattress support frame 34 are shown extending beyond the four corners of the weigh frame 30, and hence beyond the positions of the four load cells 50a-50d.

A weight distribution of a load among the plurality of load cells 50a-50d may not be the same depending on sensitivities of each of load cells 50a-50d and a position of the load on the patient support surface 36. Accordingly, a calibration constant for each of the load cells 50a-50d is established to adjust for differences in the load cells 50a-50d in response to the load. Each of the load cells 50a-50d produces a signal indicative of the load supported by that load cell 50. The loads detected by each of the respective load cells 50a-50d are adjusted using a corresponding calibration constant for the respective load cell 50a-50d. In some embodiments, the adjusted loads are then combined to establish the actual weight supported on the patient support apparatus 10. As discussed below, the signals from the load cells 50a-50d may be processed by the processor module 62 to characterize the movement of a patient into one of several classes. Thus, as configured, the bed 10 is operable as a sensor system for detecting and characterizing patient movement to provide information about the patient movement to a user either through an alarm or other communication method.

For example, six movements that patients may frequently take are considered by the processor module 62 and, when a particular movement is detected with specificity, the processor module 62 will characterize the particular movement and act on that characterization according to pre-defined protocols. The movements characterized in the illustrative embodiment include one of an action class G1-G4, where: G1 is turning right, G2 is turning left, G3 sitting up, and G4 is lying down.

Each action has its own different feature, referring to four different pressure signal expressions, so this can be defined as a classification problem. Although the time series signals P1-P4 from each respective load cell 50a-50d generated by different people doing the same kind of action in different positions are different, there are still certain characteristics and similarity that can be distinguished to characterize the action.

In the present disclosure, a convolution neural network (CNN) is applied as a framework to achieve action recognition. A group of time series signals P1-P4 from each of the respective load cells 50a-50d is processed to extract characteristics that are used as an input of the classifier in a generalized classification method. The classifications are heuristics. In the present disclosure, the signals are regarded as a two-dimensional picture and the processed signal from each load cell 50a-50d are the inputs to the CNN. Specifically, the process of convolution and the pooling filter in CNN operating along the time dimension of each sensor provides feature extraction processing, and the final output is the membership that is recognized as one of the respective actions G1-G4.

The applied CNN is a complex model where convolution and pooling (subsampling) layers alternate at typically 2-3 layers and finally get to a full connection layer through a flattening layer. The time series signals P1-P4 are used as the input of the network with limited preprocessing. Each convolution layer is equivalent to a feature extraction layer, and multiple feature maps can be obtained by different convolution kernels. In the present disclosure, the convolution neural network adopts the gradient descent method and the minimized cost function to propagate back the error layer by layer and then adjusts the weight. Using this approach the accuracy of the network to recognize the action G1-G4 is improved.

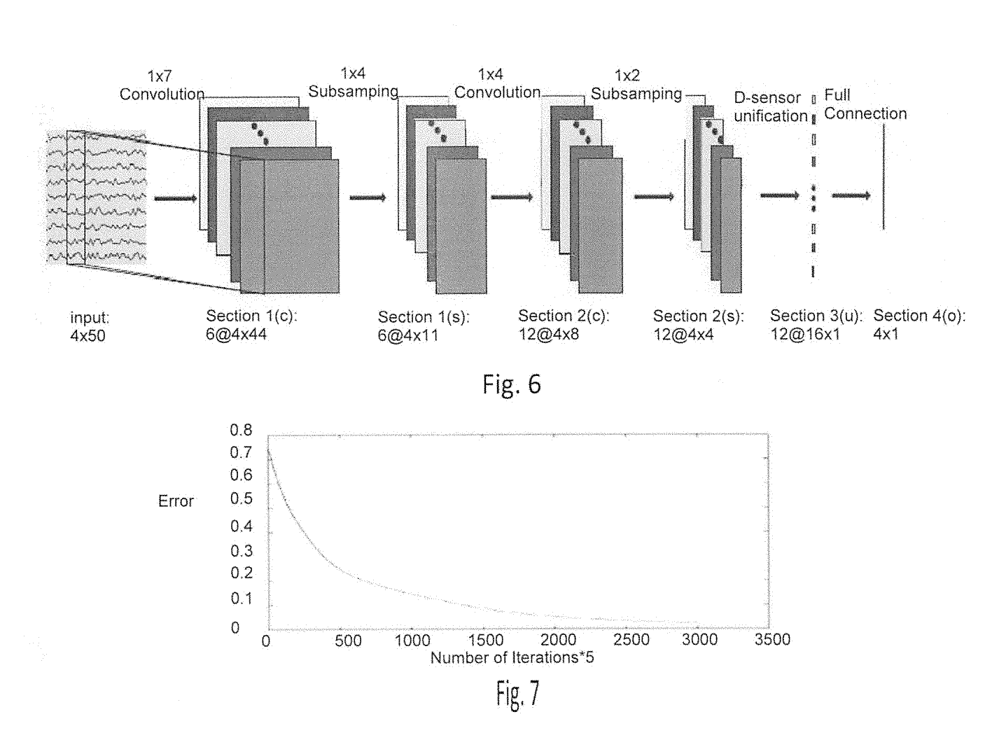

The first layer is the input layer, followed by two alternating convolution and pooling layer, to get the output of the classification after a full connection layer. The basic framework of CNN is shown in FIG. 6, wherein in the section designation, C represents a convolution layer, S represents a subsampling layer, U represents the flatting layer, and O represents the output layer. The number before and after ‘@’ refers to the number of feature maps and the dimensions of each feature map used in the CNN.

In the simplified CNN disclosed herein, the parameters applied include the batch, the learning rate η, and the convolution kernel. The batch is the number of samples for each batch of training. The size of the batch affects computing time, computing load, training time, and accuracy, as discussed below. The learning rate is multiplied by the gradient in the back propagation as an updating weight. A high learning rate can increase training speed, but the optimum can easily missed during the back propagation such that accuracy is reduced and convergence is hard to achieve. The number of convolution kernels also affects the training rate and accuracy, and the size is related to the dimension of the input. Variations in kernel size drive changes to the size of the other layers of the CNN. The stride of the kernel also affects the dimensions of each layer as well.

The approach of the present disclosure begins with data pre-processing which is applied to the signals P1-P4. Initially the amount of data noise is unknown, so it is necessary to analyze the spectrum of a plurality of data to find out whether there is a stable signal with a large difference from the signal generated by the movements G1-G4. If there is, then noise exists. After several filtering methods were tested, it was experimentally determined that a 20^{th} order median filter is appropriate for the disclosed bed 10 and load cells 50a-50d and can be described as:

y(i)=Med[x(i−N), . . . ,x(i), . . . x(i+N)] (1)
where N is set to 10, x(i) denotes the signal of current sampling point, y(i) denotes the filtered output in the i point and Med denotes to take the median value of x(i).

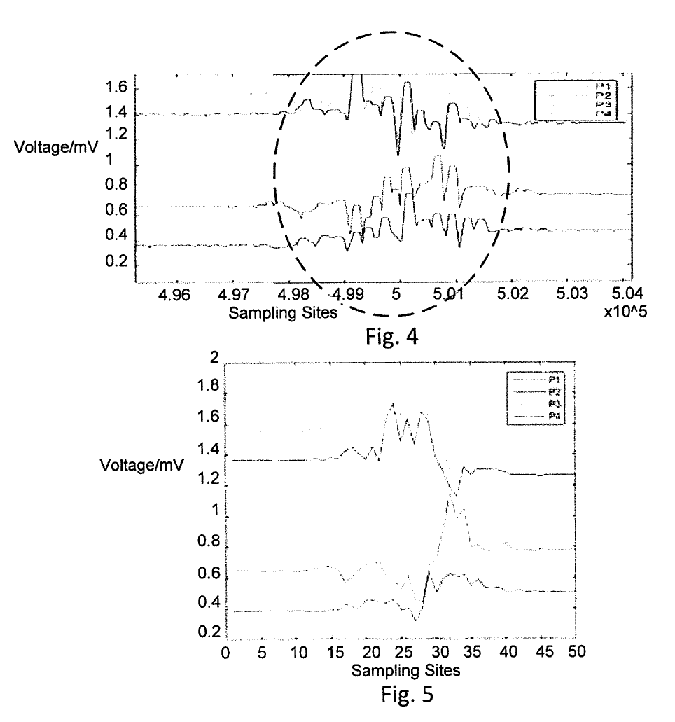

An example of a filtered signal is shown in FIG. 4: The fluctuations within the dotted ellipse in the signals P1-P4 from each of the respective load cells 50a-50d shown in FIG. 4 represents a particular movement by the occupant of the bed 10.

During training of the CNN, to develop adequate data for a high-dimensional vector, a test occupant is directed to repeat a certain action several times to get adequate data. It was determined experimentally that any of the actions G1-G4 occur in about 3-5 seconds, so 8 seconds was adopted as the sliding window length, making any action performed fully contained in an analyzed time window. Applying an 8000 Hz sampling rate results in a 4×8000 matrix. Considering that the CNN used below is not suitable for such a high-dimensional real-time input, a 4×50 matrix is established after down sampling. The down sampling preprocesses for the CNN and reduces the computation load. Using the same data as it has been presented in FIG. 4, the effect of down sampling is shown in FIG. 5.

The CNN utilizes supervised training, so the set of samples is composed of vectors such as an input vector and an ideal output vector. Initially, a random number between (−1) and 1 is used as a weight, the offset is set to 0, and the value of the convolution kernel and the bias of each layer is trained in the front propagation. The output of the convolution layer is limited to (0, 1) by the sigmoid function. Finally a 4×1 vector is obtained, and the values correspond to the memberships of each action. Each corresponding label is also a 4×1 vector where the corresponding action value is 1 and the rest are 0. The CNN is similar to a BP neural network, and it is also divided into two stages: front propagation and back propagation.

For the front propagation, the time series signals are convolved in the convolution layers with several single-dimensioned convolutional kernels (to be learned in the training process). The output of the convolution operators added by a bias (to be learned) is put through the activation function to form the feature map for the next layer. Formally, the value vᵢⱼ is given by:

vᵢⱼ=sigmoid(bᵢⱼ+Σₘconv(v₍ᵢ₋₁₎ₘ,Wᵢⱼ)) (2)
where sigmoid is the activation function, bᵢⱼ is the bias of the layer, m is the index of the (i−1)th feature map that connects to the current layer, cony is the convolution operation and Wᵢⱼ is the value of convolutional kernels.

In the pooling layers, feature maps in the previous layer are pooled over local temporal neighborhood by a sum pooling function, and the function can be described as:

$${v}_{ij}^{x , y} = \frac{1}{{Q}_{i}} {\sum }_{1 \leq q \leq {Q}_{i}} ( {v}_{( i - 1 ) j}^{x + q , y} ) \qquad ( 3 )$$

where vᵢⱼ^{x,y} is the x-th row and y-th column of the j-th feature map of the i-th layer, and Qi is length of the pooling region.

To achieve back propagation, mean square error (MSE) is used as the cost function of the neural network. The action is divided into 4 classes, so the loss function can be described as:

$${E}^{N} = \frac{1}{2} {\sum }_{n = 1}^{N} {\sum }_{k = 1}^{4} {( {t}_{k}^{n} - {y}_{k}^{n} )}^{2} \qquad ( 4 )$$

where N is the number of each batch, tₖⁿ denotes the kth dimension of the label corresponding to the nth sample, yₖⁿ denotes the kth dimension of the output corresponding to the nth sample.

There is no special regulation setting the relevant parameters for the convolution kernels. For convenience and after several attempts, the kernel number of the first layer is 6, and the size is 1×7; the second pooling layer's size is 1×4; the kernel number of the third layer is 12, and the size is 1×4; the fourth pooling layer's size is 1×2; and the convolution stride size is 1. It's noted that because the random number is selected between (−1, 1) when the weight is initialized, the effect of each training may be different. After several trainings, the average is taken.

The value of batch must be divisible by the number of samples to equally distribute all of the samples. In the illustrative implementation, a training sample of 220 groups was used, so the batch was set as 44, 22 and 11. When the learning rate is 1 and the number of iterations is 1000, accuracy and time are adopted to evaluate the performance of different batches, and the comparison results is shown in Table I:

TABLE I
Comparison between different batches

| | | | |
|---|---|---|---|
| Batch | 44 | 22 | 11 |

| | | | | |
|---|---|---|---|---|
| 1 | Accuracy | 87.0% | 83.4% | 86.3% |
| | Time | 197 s | 322 s | 568 s |
| 2 | Accuracy | 89.6% | 82.7% | 85.7% |
| | Time | 194 s | 386 s | 568 s |
| 3 | Accuracy | 86.0% | 85.3% | 84.4% |
| | Time | 192 s | 341 s | 568 s |
| Average | Accuracy | 87.5% | 83.8% | 85.5% |
| | Time | 194.3 s | 349.7 s | 568.0 s |

After comparing the three cases from Table I, accuracy is not impacted significantly by the reduction of the batch size. There is a slight decrease and the training time is significantly slowed, so the larger batch of 44 is utilized for efficiency.

After several attempts, it was experimentally determined that the learning rate between 0.5-1.5 is more appropriate. On this basis, 0.5, 1.0, 1.5 were each selected as the adaptive learning rate to compare the results. The initial value is set at the beginning and the learning rate changes as the gradient updates and the reducing the rate. Table II compares the four groups of learning rates together at a batch of 44 and the number of iterations at 1000.

Comparing the three fixed learning rates, it was found that 0.5-1.5 is a suitable range, the accuracy and training efficiency can be ensured. The Adagrad algorithm adaptively allocates different learning rates for each parameter. However it may come to a local optimal point, that is, the learning rate will not change further, as sometimes the latter part of the learning rate may be too small, and the initial learning rate is difficult to choose. Although the accuracy may be improved, the computation time is greatly increased. Considering all the factors mentioned above and empirical experience, the fixed learning rate is set to 1.

TABLE II
Comparison between Different Learning Rates

| | | | | | Initializing |
|---|---|---|---|---|---|
| Learning rate | | 0.5 | 1.0 | 1.5 | to 3 |
| 1 | Accuracy | 85.7% | 85.3% | 86.6% | 83.4% |
| | Time | 195 s | 194 s | 195 s | 222 s |
| 2 | Accuracy | 86.3% | 87.0% | 84.7% | 87.0% |
| | Time | 198 s | 195 s | 202 s | 210 s |
| 3 | Accuracy | 85.3% | 89.6% | 86.6% | 91.9% |
| | Time | 195 s | 192 s | 197 s | 255 s |
| Average | Accuracy | 85.8% | 87.3% | 86.0% | 87.4% |
| | Time | 196.0 s | 193.7 s | 198.0 s | 229.0 s |

After selecting the most suitable batch and learning rate, the number of iterations is modified making the training converge and increasing the number of iterations for improving the accuracy. After several attempts, as shown in FIG. 7, it is evident that when the number of iterations is 600, the cost function has been converged, and the accuracy is 79.2%. Then the number of iterations is gradually increased until the accuracy is more than 85%. After testing, the number of iterations was set to 800.

### Experimental Results

After selecting the relevant parameters, data was used to verify the model effect. In the illustrative approach, the system was developed using training samples, validation samples and testing samples. Testing samples were gathered through test subjects while training samples are increased by iteration. To develop the model, test subjects who are aged between 22-25 years old, weigh between 50-80 kg, and with heights distributed over 150-180 cm were employed as test subjects. Data was collected through typical data acquisition methods. The process was carried out with the data got beforehand through a certain number of repeated experiments. The test subjects were instructed to repeat the following sequence during acquisition of the signals P1-P4: [Lying]→[Turn right→Lying] (repeat)→[Turn left→Lying] (repeat)→[Sitting→Lying] (repeat).

The training samples were also taken in three locations wherein the test subject lies on the right side of the bed 10, the middle side of the bed 10 and the left side of the bed 10 respectively so that all the use cases in bed 10 can be taken into account. After pretreatment, 220 groups of training samples were acquired. In addition, 577 samples are mainly used for parameter training and membership analysis. The testing samples amount to 600 groups.

The results are as below:

TABLE III
The Test Results of the Validation Samples

| | Right | Left | Sitting | | | |
|---|---|---|---|---|---|---|
| | turning | turning | up | Lying | Sum | Accuracy |
| Right turning | 149 | 8 | 1 | 16 | 149/174 | 85.6% |
| Left turning | 7 | 171 | 8 | 11 | 171/197 | 86.8% |
| Sitting up | 2 | 10 | 115 | 2 | 100/129 | 77.5% |
| Lying | 2 | 0 | 0 | 75 | 75/77 | 97.4% |

| | |
|---|---|
| Accuracy | 88.0% |

The numbers in columns 2-4 in Table III represent the number of actions performed in the first column that are characterized as the action in that column. For example, the first line indicates that 149 right turn actions are classified correctly in 174 samples. In the remaining samples, eight are incorrectly classified as left turning, one is incorrectly classified as sitting up, and sixteen are incorrectly classified as lying. Looking in total, the incorrect classifications of the right or left turning cases are mostly divided into lying. Also, the left turning and siting sometimes will be classified wrongly while the lying is almost right. In general, the system has 88% accuracy.

The final output of the neural network is the membership of each action. After analyzing the membership, it is found that the membership is almost the same when right turning is classified wrongly as lying, which means that the probability of right turning is slightly less than lying, so it is classified as lying.

The original data was analyzed to find that in several samples there are 1-2 signals that have minimal changes. This is different from a situation where two sensor signals increase and the other sensor signals decrease when people turn over. One hypothesis is that occupants turn over without moving, leading to no obvious changes of the signals; Secondly, because of the initial weight, that is, despite the training accuracy, the situation may be slightly different, and the errors do not exist after re-training. In follow-up experiments, the accuracy greatly improves as the actions are more standard. Considering the left turning and lying, it was found that there are two columns of signals changing similarly between the left turning and sitting. Because of the non-standard left turning, a column of signals may not change clearly, then it leads to the wrong classification.

The membership mentioned in this disclosure is between 0 and 1, which is the final output of the CNN and the output is a 4×1 vector. The criterion for judging an action is to observe whether the maximum value of membership is corresponding to the label. For example, if the output is [0.7, 0.2, 0.2, 0.1] and the label is [1, 0, 0, 0], then it's classified as the right turning and it is correct. However, when analyzing the misjudgments there exists the following case: the output is [0.3, 0.1, 0.2, 0.1], and the label is [1, 0, 0, 0], then it'll be classified as the right turning as the criterion, but obviously this judgment is wrong. There are two main reasons for this situation, on the one hand, the action may not belong to the current classification of any action, that is, unclassified action; on the other hand, the action is not complete, such as turning over to half or siting up after turning over immediately. Therefore, it is necessary to analyze the membership of each action, get the probability density distribution and calculate its probability distribution to determine a confidence interval with a certain confidence.

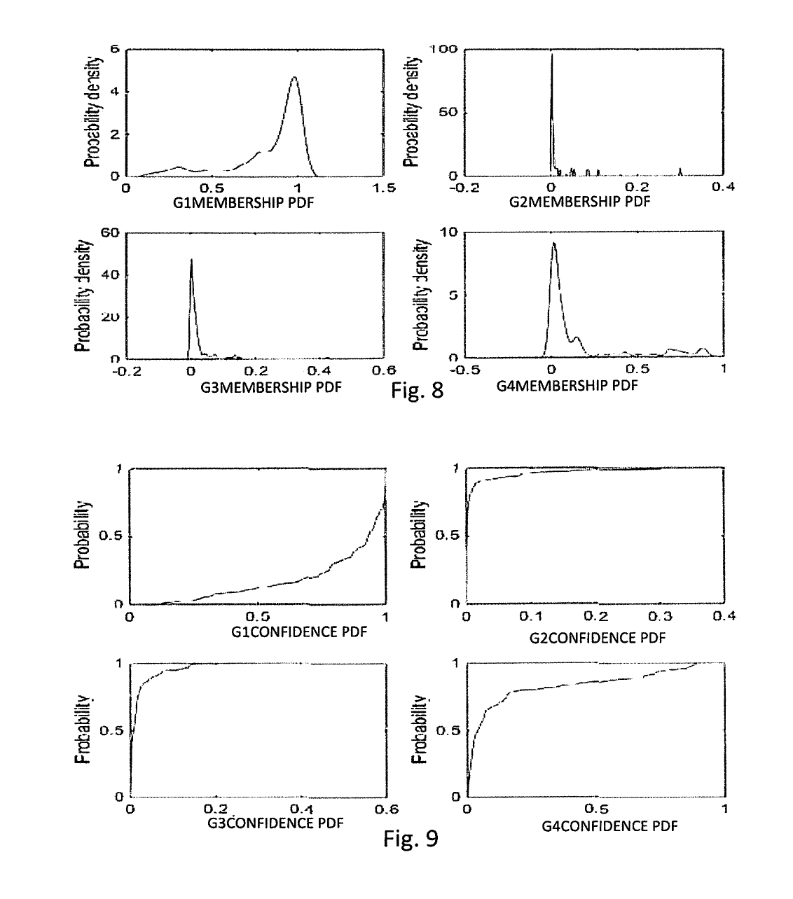

Taking the judgment of the right turning as an example, making a probability density distribution, provides the results of FIG. 8. To determine a confidence, the probability distribution function is developed as shown in FIG. 9. After comparing the four probability maps, the confidence is set to 0.85 and then the one-sided confidence interval is calculated, and the interval of membership of each action is shown as Table IV.

TABLE IV
Criteria for judgment of probability distribution of membership

| | Right | Left | Sitting | |
|---|---|---|---|---|
| | turning | turning | up | Lying |
| 1 | 0.64-1.00 | 0.00-0.09 | 0.00-0.01 | 0.00-0.09 |
| 2 | 0.00-0.02 | 0.81-1.00 | 0.00-0.08 | 0.00-0.11 |
| 3 | 0.00-0.03 | 0.00-0.04 | 0.84-1.00 | 0.00-0.04 |
| 4 | 0.00-0.43 | 0.00-0.23 | 0.00-0.05 | 0.65-1.00 |

The test samples were evaluated following the standard, and the results are summarized in Table V.

It can be found that comparing to the validation samples, although the body sizes of test subjects is different, an ideal effect is achieved after normalizing the weight. A part of sample can reach to a high accuracy, even to 100%. This confirms the applicability of the neural network to making the classification of the movement.

TABLE V
Testing results

| | Right | Left | Sitting | | |
|---|---|---|---|---|---|
| | turning | turning | up | Lying | Accuracy |
| Test subject1 | 18/18 | 20/20 | 10/10 | 12/12 | 100% |
| Test subject2 | 14/14 | 18/18 | 17/17 | 11/11 | 100% |
| Test subject3 | 18/18 | 19/19 | 20/20 | 13/13 | 100% |
| Test subject4 | 20/20 | 19/20 | 20/20 | 10/10 | 98.6% |
| Test subject5 | 19/20 | 14/18 | 10/14 | 18/18 | 87.1% |
| Test subject6 | 16/16 | 16/16 | 20/20 | 18/18 | 100% |
| Test subject7 | 22/22 | 17/18 | 16/16 | 7/14 | 88.6% |
| Test subject8 | 20/20 | 18/18 | 19/19 | 13/13 | 100% |
| Test subject9 | 17/17 | 14/14 | 17/17 | 21/22 | 98.6% |
| Sum | 164/165 | 155/161 | 149/153 | 113/121 | 96.8% |

In the illustrative embodiment, the determination of the particular classification as G1-G4 is tested for probability of the determination being a true condition and if the error is sufficiently small, the movement is characterized in the particular classification such that the processor module 62 signals that movement to the alarm system 90 so that a user, such as a nurse, may be notified of the movement and take corrective action. Various corrective actions may be implemented by the user/caregiver/nurse or other systems on the bed 10 may be signaled to initiate a corrective action. For example, portions of the bed 10 may be moved automatically to make the indicated movement easier for the patient.

As discussed above, machine learning algorithms use feature extraction and, in some instances, are unable to acquire the appropriate features. With a deep learning approach, the complexity drives ever increasing number of parameters for training and requires time for training to learn.

Another approach that may be used is an activity recognition method based on the random vector functional-link neural network (RVFLNN). The training process of RVFLNN is relatively short and the model can be quickly established because of the characteristics of its network structure. In addition, RVFLNN has minimal dependence on parameters, provides improved function approximation ability, and improved generalization ability. Using data signals from the load cells 50a-50d, the data are filtered using median filtering and down sampling as discussed above with regard to the CNN embodiment. Then the preprocessed data are manipulated by a sparse auto encoder in deep learning to extract a feature. The feature is then fed into the RVFLNN to determine the ideal output. Additionally there is incremental learning for model updating. The combined feature extraction and incremental learning provides a broad learning system. As discussed below, the accuracy and training time are provide an improved response compared with the CNN approach discussed above.

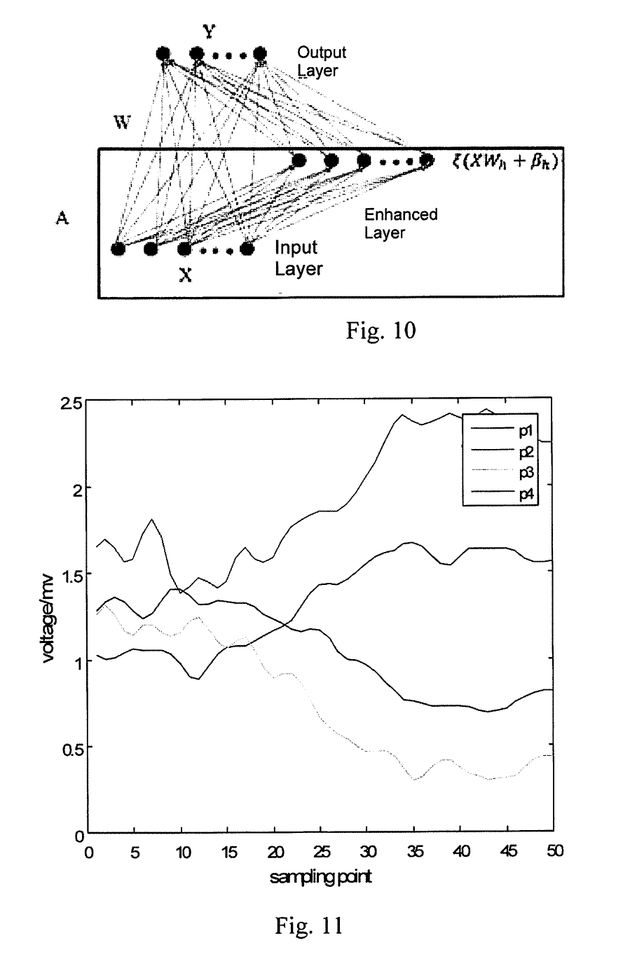

Referring to FIG. 10, the portions of a massive net can be replaced with functional-links (FL) as further described below. The FL net is a neural network that combines the hidden layer with the input layer. The RVFLNN performs a nonlinear transformation of the input pattern before it is fed to the input layer of the network.

Thus the enhanced pattern is shown as below:

E=ξ(XWₕ+βₕ) (5)
where Wₕ is a random vector, βₕ is the bias and ξ is the activation function.

The network output can be defined as the equation AW=Y where A=[X E], here the W can be quickly calculated by matrix operation instead of iterative training.

As discussed above with regard to the CNN embodiment, action recognition is implemented using a neural network. The single layer forward neural network (SLFN) is widely used for classification or regression and the traditional method of training neural networks is gradient descent, but it is relatively easy to fall into local minimum or overfitting, and often the network needs to be retrained with new sample. The RVFLNN is an alternative approach that reduces computing power required to achieve learning.

In the present RVFLNN embodiment, the 20^{th} order median filtering and pre-processing is applied as discussed above. To facilitate the learning, raw data signals are collected over a period of time. Learning is accomplished by evaluating the data signals from the same movement conducted multiple times over a sample of individuals. As the time to complete each movement varies from each individual, sliding windows for detecting the action is not practical. To overcome this variation, each sample is monitored for a large fluctuation to determine the boundary of the segmentation by the fluctuation condition. A sample starts with a large fluctuation of the signal, and end also with a follow on large fluctuation. The process proceeds by calculating whether the value at a sampling point (i+m) exceeds the value at the sample point i by more than a certain amount. If it does, then the algorithm records the sample point i, and monitors the signal after the sample point i to find another sampling point j which meets the condition that the several consecutive changes are less than a certain value. Once this determination made, the duration from point i to j is determined to be an action sample.

This approach provides that each sample size/duration is different. To perform the analysis, it's necessary to normalize the sample sized. Importantly, the sampling frequency is set to 1000 Hz and it takes several seconds to complete an action, so down sampling is significant.

After several attempts, it has been determined empirically that the appropriate size of each sample in the disclosed embodiment requires the various samples to be down sampled to be 4×50 (where 4 denotes the four load cell signals). Then the matrix is flattened by row so that it can serve as the input of the network, that is, the final input is a vector of which size is 1×200. For reference, the input without flattening is shown in FIG. 11.

Due to the increase in the amount of data, the dimensions of the data are constantly increasing. If the original data is directly fed into the neural network, under certain hardware conditions, the system may not be able to process the data. To address the data expansion, two methods may be employed. One approach is dimensionality reduction and the other is feature extraction. It has been determined that in the present embodiment, feature extraction is the most viable to find the best expression for the original sample, such as statistical features, random feature extractions such as non-adaptive random projections, principle component analysis (PCA), or feature extraction layer like convolution layer in deep learning, etc. Taking into account the fact that the neural network is not suitable for discrete statistical features, it has been determined that a sparse auto encoder in deep learning is viable to complete feature extraction.

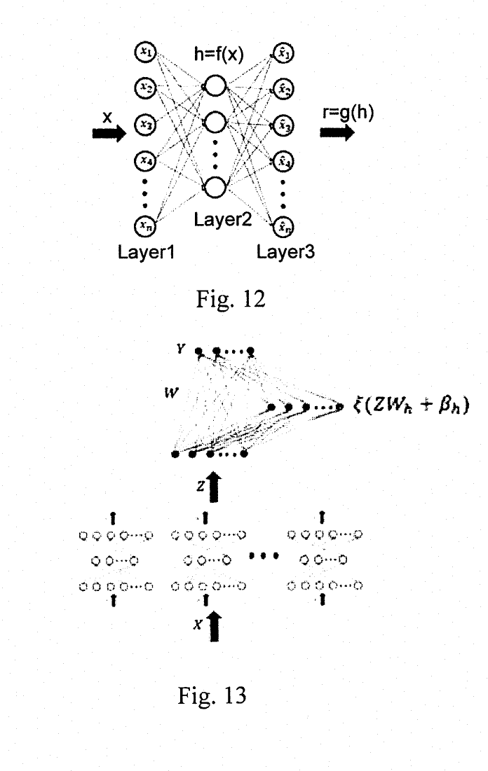

Sparse auto encoder is a type of neural network that can be trained to copy input to the output. A simplified auto encoder structure is shown in FIG. 12. Since the auto encoder has a hidden layer h inside, it can generate the representation of input. The network consists of two parts: an encoder represented by the function h=f(x) and a decoder represented by the function r=g(h). The auto encoder must be constrained, therefore it can only be replicated approximately. The constraints force the model to consider which parts of the input data need to be copied to allow it to learn useful characteristics of the data.

In general, the hidden layer nodes of the auto encoder are smaller than the number of input nodes, but may also be larger than the number of input nodes in some embodiments. Defining a particular sparsity limit can achieve the same effect. The encoder is sparse in that most of the nodes in the hidden layer are suppressed while a small part are activated. If the nonlinear function is a sigmoid function, it is active when the output of the neuron is close to 1, and sparse when it is close to 0; if the tan h function is used, it is activated when the output of the neuron is close to 1, and is sparse when it is close to −1.

This embodiment uses the relative entropy Kullback-Leibler divergence (KL divergence) so that the activity of the hidden layer nodes is very small [17]. KL's expression is as below:

$$KL ( ρ ‖ {\hat{ρ}}_{1} ) = ρ log \frac{ρ}{{ρ}_{j}} + ( 1 - ρ ) log \frac{1 - ρ}{1 - {\hat{ρ}}_{j}} \qquad ( 6 )$$

where ρ is called the sparse parameter which is set to be relatively small and \hat{ρ}ⱼ is defined as below:

$${\hat{ρ}}_{j} = \frac{1}{m} {\sum }_{i = 1}^{m} {a}_{j}^{2} ( {x}^{i} ) \qquad ( 7 )$$

where aⱼ²(x) denotes the activation of the hidden neuron j of x, and m is the number of the data.

And the loss function without sparse can be given by:

$$J = [ \frac{1}{2 m} {\sum }_{i = 1}^{m} {‖ g ( f ( {x}^{i} ) ) - {y}^{i} ‖}_{2}^{2} ] + \frac{λ}{2} ( {‖ {W}_{e} ‖}_{2}^{2} + {‖ {W}_{d} ‖}_{2}^{2} ) \qquad ( 8 )$$

where y is the label, Wₑ is a weight matrix of the encoder, W_{d} is the weight matrix of the decoder and λ is used to control the strength of the penalty term.

Thus the final loss function is shown as below:

$${J}_{sparse} = J + β {\sum }_{j = 1}^{s} KL ( ρ ‖ {\hat{ρ}}_{j} ) \qquad ( 9 )$$

where β controls the strength of the sparse term and s is the number of the output nodes.

With the loss function defined, the random vector W for equation 5 can be determined by gradient descent just like a neural network, completing the feature extraction.

Combined with feature extraction and incremental learning, the broad learning system is proposed to meet better performance and scalability as illustrated in FIG. 13.

In the broad learning system, first, the feature map Z=[Z₁, . . . , Zᵢ] is produced from the x. And the Zᵢ is given by:

Zᵢ=ϕ(XWₑₜ+βₑ) (10)
where ϕ is an activation function, Wₑₜ is the weight of encoder in sparse auto encoder and β is the relative bias.

Then, randomly generated weights Wₕⱼ are used on the feature map to obtain enhancement nodes H=[H₁, . . . , Hⱼ], where Hⱼ is given by:

Hⱼ=ψ(ZWₕⱼ+βₕ) (11)

Finally, the feature map Z and enhancement nodes H are concatenated and then fed into the output returning to the basic equation AW=Y, and a pseudoinverse, such as the Moore-Penrose matrix inverse, is a very convenient approach to solve the output-layer weights of a neural network. The traditional matrix operation solution is to get the pseudoinverse by singular-value decomposition (SVD). However, it may affect the efficiency and not work well in the case of a large amount of data, so an approximate method is used here to solve the question. In this embodiment, the optimization function is convex and has a better generalization performance is as defined in Equation 12 below:

$$arg {min}_{W} \text{:} {‖ AW - Y ‖}_{2}^{2} + λ {‖ W ‖}_{2}^{2} \qquad ( 12 )$$

where a L2 norm regularization is added to lower the complexity of the network and prevents overfitting and the λ is set to control the strength of the L2 norm regularization. Then this solution is equivalent with the ridge regression theory, so the solution can be determined as shown in Equation 13 below:

W=(λI+A^{T}A)⁻¹A^{T}Y (13)

Then, as an approximation:

A⁺=(λI+A^{T}A)⁻¹A^{T} (14)

In various applications, although there is a process of feature extraction and enhancement node, sometimes the accuracy may not satisfy the requirements without a corresponding training process. Similar to traditional neural networks, this broad learning system also requires increasing the number of nodes.

The ordinary neural networks or deep neural network such CNN and recurrent neural network (RNN) have a training process. The training process takes time and resources, especially when there are new samples, or the number of categories in the classification problem changes, or the network structure needs to be modified. In these cases, the previously trained model cannot be used, and it needs to be retrained, which is time consuming. Thus, the incremental method in this embodiment of broad learning system is used to adjust the weight without retraining the whole network. There are two cases considered here for incremental learning.

First, there are new input data. To address the new data, the old model is denoted as AₙWₙ=Y, and the new input data is X. The same feature extraction and enhancement are used in the input, then Aₓ is determined, and the update input is as below:

$${A}_{n + 1} = [ \begin{matrix}{A}_{n} \\ {A}_{x}^{T}\end{matrix} ] \qquad ( 15 )$$

At this point, the matrix pseudoinverse can be calculated as follows:

$$\begin{matrix}{A}_{n + 1}^{+} = [ {A}_{n}^{+} - {BD}^{T} B ] \\ where {D}^{T} = {A}_{x}^{T} {A}_{n}^{+} & ( 16 ) \\ B = \{ \begin{matrix}{C}^{+} & if c \neq 0 \\ {( 1 + {D}^{T} D )}^{- 1} {A}_{n}^{+} D & if c = 0\end{matrix} \\ and C = {A}_{x}^{T} - {D}^{T} {A}_{n} & ( 17 )\end{matrix}$$

Therefore the updated weights are

Wₙ₊₁=Wₙ+B(Yₓ^{T}−Aₓ^{T}Wₙ) (18)

In the second case for incremental learning, consider the case that the number of classifications needs change with new categories available. For classification, One-Hot encoding is used as label which means that the vector [1, 0, 0, 0, 0, 0] corresponds to an action. So when new categories are needed, the new label Yₙ₊₁ can be defined as:

$${Y}_{n + 1} = [ \begin{matrix}{Y}_{n} & 0 \\ {Y}_{a} & \end{matrix} ] \qquad ( 19 )$$

Therefore the updated weights are

Wₙ₊₁=Aₙ₊₁⁺Yₙ₊₁ (20)

TABLE VI
Classification Results of broad learning system

| | Actions | | | | | |
|---|---|---|---|---|---|---|
| | | Turn | Turn | Stretch | | |
| | | over to | over to | out for | Sit | Lie |
| Results | Exit | left | right | something. | up | down |
| Exit | 31 | 0 | 0 | 0 | 0 | 0 |
| Turn over to left | 0 | 71 | 0 | 0 | 1 | 0 |
| Turn over to right | 0 | 0 | 39 | 0 | 2 | 0 |
| Stretch out for | 0 | 0 | 1 | 29 | 0 | 0 |
| something. | | | | | | |
| Sit up | 0 | 0 | 0 | 0 | 60 | 0 |
| Lie down | 0 | 0 | 0 | 0 | 0 | 67 |
| Accuracy (100%) | 100 | 100 | 97.50 | 100 | 95.24 | 100 |

### Experimental Results

In a neural network, the training samples are needed for training the network and testing samples are for testing the accuracy. Testing samples are gathered through volunteers while training samples can be increased by iteration. In addition, some samples split from the volunteer samples are added into training samples for the generalization. In an implementation of the broad learning model using RVFLNN, ten volunteers who are mainly undergraduate and graduate students, aged between 22-25 years old, weigh between 50-80 kg, and having heights distributed in 150-180 cm were used. In this model two additional movements were added to the four discussed in the CNN model above. The six kinds of bed-related actions that patients may frequently take are chosen for analysis, which are designed by an experienced nurse are turning over to left, turning over to right, sitting up, lying down, stretching out for something and exiting from the bed. For training samples, the process is carried out with the data got beforehand through a certain number of repeated cycles.

Upon starting the signal acquisition, and the movement sequence followed is as below:

[Lying] →[Turn right→Lying] (repeat)→[Turn left→Lying] (repeat)→[Sitting→Lying] (repeat)→[Stretching out→Lying] (repeat)→[Exiting→Lying] (repeat)

It should be noted that in a real daily situation in hospital, the intensity and speed of each action may vary from one patient to another. For bedridden patients, elderly patients or critically ill patients, their motion patterns are mostly static-action-static, that is, it is difficult for them to continuously complete such actions as sitting up, lying down, turning over, etc. for a period of time. So there will be a relatively quiet time as a transition. Thus the volunteers were asked to lie on the bed for some time and do whatever action they like. At last, over 1000 samples are obtained for the network and the sample proportion is about 3:1.

In the final experimental sample, there are 790 training samples, 301 test samples, and 460 samples for incremental samples. To determine the viability of the broad learning approach, final experimental results are mainly compared with the CNN approach discussed above.

The results are shown in Table VI and Table VII. It can be seen that the original data themselves are not very complex, and after a certain preprocessing, paying attention to the adjustment of the parameters in the training process, the rational use of the activation function, and shuffling data before training, standardization, etc. both networks have achieved a good result, which also confirms that the neural network can be well applied to activity recognition.

TABLE VII
Classification Results comparison of broad
learning system and deep learning

| | | |
|---|---|---|
| Method | Training time | Accuracy(test) |

| | | | |
|---|---|---|---|
| Broad learning | 0.12 | s | 0.9834 |
| system | | | |
| Deep | 3 | min | 0.9933 |
| learning(CNN) | | | |

When comparing the accuracy, it can be seen that the complexity of the network will also affect the accuracy to a certain extent. The more complex it is, the deeper it is. The convolutional layer is a process of feature extraction, through several alternating convolution and pooling, a relatively good effect is finally obtained. However, the problem is that with the complexity of the network, the training is difficult; it time-consuming to use the gradient descent method to train the network compared to the direct solution. It can be seen from Table VII that the broad learning system uses only 0.12 s, with excellent accuracy.

Referring now to Table VIII, when there are new 460 samples, the convolutional neural network will inevitably need to be retrained, which, in the illustrative embodiment, takes 5 minutes while the training time is still relatively larger than broad learning system of 0.07 s. As for the accuracy, the CNN still achieves a high precision, and there is a certain reduction in accuracy but within acceptable limits. Therefore, if the accuracy is very important, then the deep learning CNN is more appropriate, if there is hardware or other resource constraints or that model often needs to be updated, and the accuracy requirement is not strict, then the broad learning system is a viable alternative.

TABLE VIII
Comparison of broad learning system and deep
learning with increment for classification

| | | |
|---|---|---|
| Method | Training time | Accuracy(test) |

| | | | |
|---|---|---|---|
| Broad learning | 0.07 | s | 0.9636 |
| system | | | |
| Deep | 5 | min | 0.9900 |
| learning(CNN) | | | |

In summary, a data-driven human activity classification method based on load cell signals for a patient support apparatus can be accomplished with excellent accuracy. With the processing of the original signals and sparse auto encoder for feature extraction, the classification model using broad learning system achieves viable results. Compared with the CNN in deep learning, the training time is greatly reduced when the accuracy is very high. In addition, the incremental learning is considered with new samples available and new categories is less taxing on system resources as compared to the deep learning CNN. The deep learning model has to be retrained with calculation burden and time cost. In comparison, using the broad learning model, the network weights can be directly updated without retraining the entire model, and the accuracy is also guaranteed, revealing superiority for activity recognition for patients in bed.

Although this disclosure refers to specific embodiments, it will be understood by those skilled in the art that various changes in form and detail may be made without departing from the subject matter set forth in the accompanying claims.

1. A sensing system for detecting, classifying, and responding to a patient action comprising
a frame,
a plurality of load sensors supported from the frame,
a patient supporting platform supported from the plurality of load sensors so that the entire load supported on the patient supporting platform is transferred to the plurality of load sensors,
a controller supported on the frame, the controller electrically coupled to the load sensors and operable to receive a signal from each of the plurality of load sensors with each load sensor signal representative of a load supported by the respective load sensor, the controller including a processor and a memory device, the memory device including a non-transitory portion storing instructions that, when executed by the processor, cause the controller to:
capture time sequenced signals from the load cells,
input the time sequenced signals to a convolution neural network to establish the membership of an action indicated by the signals,
apply a probability density function to the membership determination to establish a confidence interval for the particular membership, and
if the confidence is sufficient, provide an indicator identifying the most likely action indicated by the signals.

2. The sensing system of claim 1, wherein the time sequenced signals are filtered using median filtering applied to predefined groups of time sequenced data points of the signals.

3. The sensing system of claim 2, wherein the filtered data signals are down sampled prior to being input into the convolution neural network.

4. The sensing system of claim 3, wherein the convolution neural network is trained using historical signal data.

5. The sensing system of claim 4, wherein the output of the convolution neural network is limited to either a value of 0 or 1 using the sigmoid function.

6. The sensing system of claim 5, wherein the feature map of a convolution layer output is pooled over a local temporal neighborhood by a sum pooling function.

7. The sensing system of claim 1, wherein a mean square error function is applied as a cost function for the neural network.

8. The sensing system of claim 1, wherein the load signals are normalized based on the patient's weight.

9. A method of operating a sensing system for detecting, classifying, and responding to a patient action on a patient support apparatus comprising
capturing time sequenced signals from load cells supporting a patient,
inputting the time sequenced signals to a convolution neural network to establish the membership of an action indicated by the signals,
applying a probability density function to the membership determination to establish a confidence interval for the particular membership, and
if the confidence is sufficient, providing an indicator identifying the most likely action indicated by the signals.

10. The method of claim 9, wherein the time sequenced signals are filtered using a median filter applied to predefined groups of time sequenced data points of the signals.

11. The method of claim 10, wherein the filtered data signals are down sampled prior to being input into the convolution neural network.

12. The sensing system of claim 9, wherein the convolution neural network is trained using historical signal data.

13. The sensing system of claim 9, wherein the output of the convolution neural network is limited to either a value of 0 or 1 using the sigmoid function.

14. The sensing system of claim 13, wherein the feature map of a convolution layer output is pooled over a local temporal neighborhood by a sum pooling function.

15. The sensing system of claim 9, wherein a mean square error function is applied as a cost function for the neural network.

16. The sensing system of claim 9, wherein the load signals are normalized based on the patient's weight.

17. A sensing system for detecting, classifying, and responding to a patient action comprising
a frame,
a plurality of load sensors supported from the frame,
a patient supporting platform supported from the plurality of load sensors so that the entire load supported on the patient supporting platform is transferred to the plurality of load sensors,
a controller supported on the frame, the controller electrically coupled to the load sensors and operable to receive a signal from each of the plurality of load sensors with each load sensor signal representative of a load supported by the respective load sensor, the controller including a processor and a memory device, the memory device including a non-transitory portion storing instructions that, when executed by the processor, cause the controller to:
capture time sequenced signals from the load cells,
input the time sequenced signals to a broad learning network to establish the classification of an action indicated by the signals, and
provide an indicator identifying the most likely action indicated by the signals.

18. The sensing system of claim 17, wherein the time sequenced signals are filtered using a median filter applied to predefined groups of time sequenced data points of the signals.

19. The sensing system of claim 18, wherein the filtered data signals are down sampled prior to being input into the broad learning network.

20. The sensing system of any of claim 18, wherein the broad learning network includes a sparse auto encoder for feature extraction.

21. The sensing system of any of claim 19, wherein the broad learning network includes a random vector functional-link neural network for classification of the action.

22. The sensing system of any of claim 21, wherein the sparse auto-encoder utilizes a sigmoid function to determine the activation of the neurons of the neural network.

23. The sensing system of any of claim 21, wherein the sparse auto-encoder utilizes a tangent function to determine the activation of the neurons of the neural network.

24. The sensing system of any of claim 21, wherein the sparse auto-encoder utilizes the Kullback-Leibler divergence method to determine the activation of the neurons of the neural network.

25. The sensing system of any of claim 21, wherein the random vector is determined by gradient descent.

26. The sensing system of any of claim 21, wherein enhancement nodes of the neural network are determined using randomly generated weights on the feature map.

27. The sensing system of any of claim 21, wherein the pseudoinverse of the feature matrix is determined by a convex optimization function.
